(19) United States
(12) Patent Application Publication
(10) Pub. No.: US 2007/0105780 A1
(43) Pub. Date: May 10, 2007

(54) Therapeutic agent for iNOS generating illness

(76) Inventors: Robert J. Webber, Las Vegas, NV (US); Douglas S. Webber, Los Angeles, CA (US)

Correspondence Address: THEODORE J. BIELEN JR., 1390 WILLOW PASS ROAD, SUITE 1020, CONCORD, CA, 94520, US

(21) Appl. No.: 11/268,946

(22) Filed: Nov. 7, 2005

(51) Int. Cl. A61K 38/10

(52) U.S. Cl. 514/15

(57) ABSTRACT

Treatment of SIRS, sepsis, severe sepsis and septic shock in a mammalian subject in which iNOS is present by decreasing the proteolytic cleavage of the iNOS protein using a protease inhibitor is described.

### BACKGROUND OF THE INVENTION

The present invention relates to a novel and useful therapeutic agent for the treatment for systemic inflammatory response syndrome (SIRS) which is also called pre-sepsis, sepsis, severe sepsis, and septic shock based upon inhibition of the proteolytic cleavage of inducible nitric oxide synthase (iNOS).

Nitric oxide synthase (NOS) is an enzyme which is found in humans and other mammals. Three isoforms of NOS have been identified. In the mammalian body nNOS and eNOS are constitutively expressed in the cells in which they are found. However, iNOS is not constitutively expressed, but is known to be induced by a number of cytokines, lipopolysaccharides (LPS), and other mediators of the inflammatory response. Specifically, iNOS has been associated with certain pathologies. Notably, iNOS in the blood heralds the onset of sepsis, severe sepsis, and septic shock in humans. Sepsis is estimated to kill more than 250,000 people annually in the United States alone. Of the people who develop sepsis, approximately thirty percent die from this pathophysiology.

Reference is made to U.S. Pat. No. 6,531,578 in which monoclonal antibodies are described that are specific for the recognition of iNOS in humans without cross-reacting with human eNOS or nNOS. An immunoassay using such monoclonal antibodies is capable of detecting iNOS in the blood which indicates the presence of sepsis within a very short period of time, a matter of minutes, when compared to the prior art tests which required several days to complete and obtain results.

If sepsis is treated aggressively after recognition of its existence, persons afflicted with sepsis have a much better chance of recovering and surviving. Treatment of sepsis has been limited to known antibacterial, antifungal, and antiviral treatments. Such treatments have achieved limited success even with the rapid recognition of the presence of sepsis in a human.

U.S. patent application Ser. Nos. 10/849,768 and 11/129,452 describe therapies of this type based on the analysis of more than 1200 plasma samples obtained from ICU patients with sepsis or severe sepsis or at risk for developing sepsis as part of a clinical study for a potential new diagnostic for sepsis. The presence of iNOS in the plasma of patients with sepsis and those who would become septic within the next 48 hours was discovered. Also, particulate or vesicle associated iNOS was discovered in the plasma of septic patients. Neither soluble nor particulate iNOS was found in normal controls or in ICU patients who were not septic, such as trauma patients that often display similar physiological signs and symptoms as septic patients. It was later hypothesized that particulate or vesicle-associate iNOS was partially responsible for the pathophysiology of sepsis and that the removal or neutralization of the particulate or vesicle associated iNOS might improve the outcome of septic patients.

Further, it was found that iNOS immunoreactive cell fragments or vesicles in addition to intact iNOS were present in the plasma of patients with sepsis but not in the plasma of non-septic patients. Based upon these observations, it was hypothesized (1) that the conversion from soluble iNOS to particulate iNOS might result, at least in part, from proteolytic cleavage of the iNOS protein; (2) that stopping the proteolytic cleavage of iNOS through the use of a protease inhibitor might decrease the lethality of the particulate iNOS fraction; (3) that lower molecular weight fragments of iNOS might contribute to the pathology of SIRS, sepsis, severe sepsis and septic shock; and (4) that inhibition of the proteolytic cleavage of iNOS might be a beneficial therapy for the treatment of patients with or at risk for developing SIRS, sepsis, severe sepsis and septic shock.

An article entitled “Cloning and Characterization of Inducible Nitric Oxide Synthase from Mouse Macrophages”, Xie et al, Science, 256: 225-228 (1992), reported the cloning and isolation of iNOS. The iNOS enzyme was described as a soluble cytoplasmic protein.

Subsequently, articles entitled “Nitric Oxide: Novel Biology with Clinical Relevance”, Billiar, Ann Surg, 221#4: 339-349 (1995); “Nitric Oxide: Pathophysiological Mechanisms”, Gross et al, Annu Rev Physiol, 57: 737-769 (1995); “The Cell Wall Components Peptidoglycan and Lipoteichoic Acid from Staphylococcus Aureus Act in Synergy to Cause Shock and Multiple Organ Failure”, De Kimpe et al, Proc Natl Acad Sci USA, 92: 10359-10363 (1995); “Mechanism of Gram-Positive Shock: Identification of Peptidoglycan and Lipoteichoic Acid Moieties Essential in the Induction of Nitric Oxide Synthase, Shock, and Multiple Organ Failure”, Kengatharan et al, J Exp Med, 188#2: 305-315 (1998); and “Induction of Nitric Oxide Synthase in RAW 264.7 Macrophages by Lipoteichoic Acid from Staphylococcus aureus: Involvement of Protein Kinase C— and Nuclear Factor-κB-Dependent Mechanisms”, Kuo et al, J Biomed Sci, 10: 136-145 (2003), point to the fact that the lipopolysaccharide (LPS) cell-wall component of gram-negative bacteria, the lipoteichoic acid and peptidoglycan cell-wall components of gram-positive bacteria, fungi, and viruses, can induce iNOS expression in vivo and in vitro in a wide variety of cell types.

Articles entitled-“Mechanisms Of Suppression Of Macrophage Nitric Oxide Release By Transforming Growth Factor Beta”, Vodovotz et al, J Exp Med, 178#2: 605-613 (1993); “Vesicle Membrane Association Of Nitric Oxide Synthase In Primary Mouse Macrophages”, Vodovotz et al, J Immunol, 154#6: 2914-2925 (1995); and “Bladder Instillation And Intraperitoneal Injection Of Escherichia coli Lipopolysaccharide Up-Regulate Cytokines And iNOS In Rat Urinary Bladder”, Olsson et al, J Pharmacol Exp Ther, 284#3: 1203-1208 (1998), have shown that since the discovery of iNOS in mouse macrophages, its intracellular location is not exclusively in the cytosol. Vesicle-associated iNOS has been recognized and reported.

Articles entitled “Caveolin-1 Down-Regulates Inducible Nitric Oxide Synthase Via The Proteasome Pathway In Human Colon Carcinoma Cells”, Felley-Bosco E et al, Proc Natl Acad Sci USA, 97#26: 14334-14339 (2000); “Macrophage Nitric Oxide Synthase Associates With Cortical Actin But Is Not Recruited To Phagosomes”, Infect Immun, Webb J L et al, 69#10: 6391-6400 (2001); “Epithelial Inducible Nitric-Oxide Synthase Is An Apical EBP50-Binding Protein That Directs Vectorial Nitric Oxide Output”, Glynne P A et al, J Biol Chem, 277#36: 33132-33138 (2002); “Caveolin-1-Mediated Post-Transcriptional Regulation Of Inducible Nitric Oxide Synthase In Human Colon Carcinoma Cells”, Felley-Bosco E, Biol Res, 35#2: 169-176 (2002); “Heat Shock Protein 90 As An Endogenous Protein Enhancer Of Inducible Nitric-Oxide Synthase”, Yoshide M et al, J Biol Chem, 278#38: 36953-36958 (2003); “Protein Interactions With Nitric Oxide Synthase: Controlling The Right Time, The Right Place, And The Right Amount Of Nitric Oxide”, Kone B C et al, Am J Physiol Renal Physiol, 285#2: F178-F190 (2003); and “Protein-Protein Interactions Involving Inducible Nitric Oxide Synthase”, Zhang W et al, Acta Physiol Scand, 179#2: 137-142 (1997), have reported that when induced cells are lysed and fractionated by centrifugation, iNOS is found in the particulate fraction, and that iNOS has been found associated with a number of other proteins through protein-protein interaction. Such protein-protein interactions (other proteins associated with iNOS) include cortical actin, EBP 50 (ezrin-redixin-moesin-binding phosphoprotein 50), caviolin-1, Hsp90 (heat shock protein 90), kalirin, NAP110 (NOS-associated protein 1.10 kd), and Rac-GTPases. These protein-protein interactions have been found to localize iNOS to specific regions or structures within cells. Upon cell lysis and fractionation by centrifugation, either through vesicle association or by protein-protein interaction, a portion of the supposedly soluble iNOS protein has been shown to partition into the particulate fraction.

U.S. patent application Ser. No. 09/628,585, revealed the fact that iNOS found free in the liquid portion of the blood of a patient, i.e. plasma, indicates such patient has sepsis or will develop sepsis within the next 24 to 48 hours. Using a very sensitive chemiluminescent sandwich enzyme immunoassay (EIA), such plasma iNOS can be used as a very specific biochemical marker for the onset of sepsis. The heretofore referenced chemiluminescent sandwich (EIA) was based upon two of the anti-iNOS monoclonal antibodies (MAbs) of a panel of anti-iNOS antibodies which are disclosed in U.S. Pat. No. 6,531,578, mentioned heretofore.

In an article entitled “Transcriptional Regulation of Human Inducible Nitric Oxide Synthase Gene In An Intestinal Epithelial Cell Line” Linn et al, Am J. Physiology, 272; G1499-G1508 (1997), it was shown that DLD-1 cells can be induced to produce human iNOS.

In an article entitled “Mechanisms of Suppression of Inducible Nitric-oxide Synthase (iNOS) Expression in Interferon (IFN)-γ-stimulated RAW 264.7 Cells by Dexamethasone”, Walker, G. et al, the Journal of Biological Chemistry Vol. 272, No. 26, June 1997, pp. 16679-16687; it was indicated that the glucocoticoid analogue dexamethasone suppresses iNOS formation in situ at different levels of gene expression in IFNγ-induced RAW 264.7 cells, a mouse macrophage cell line. It was indicated in this article that a reduction in the transcription rate of the iNOS gene and a decrease in the stability of the iNOS mRNA caused an approximate 50% reduction in the steady state level of the iNOS mRNA. Also in the presence of dexamethasone, two post-translational mechanisms resulted in a decreased level of the iNOS protein: (1) decreased translation of the iNOS mRNA and (2) increased protein degradation. The protease responsible for the cleavage of iNOS in the presence of dexamethasone was identified as calpain, a neutral cysteine protease. Two iNOS bands were observed in western blot analyses, but only the upper band at 130 kDa was used for quantitative analyses.

An article entitled “Proteolytic Cleavage of Inducible Nitric Oxide Synthase (iNOS) by calpain I”, Walker, G. et al, Biochimica et Biophysica Acta pp 216-224, 2001, showed that calpain cleavage of scissile bonds in the iNOS protein occurred at the calmodulin binding site and a distinct conformational site contained within the iNOS protein. Inhibition of the enzymatic activity of calpain in the supernatant fraction of the dexamethasone treated, induced cell lysate caused a reduction in the amount of cleavage of recombinant iNOS protein. Specific inhibitors, such as E-64, Ac-Leu-Leu-NLeu-al, and leupeptin were used to inhibit such cleavage. Calpain was shown only to cleave the iNOS monomer and not the ezymatically active dimeric form of iNOS. The calpain inhibitor I used in these experiments also inhibited the proteolytic activity of cathpsins B and L and of the proteasomal degradation pathway.

An article entitled “Inducible Nitric-oxide Synthase Is Regulated by the Proteasome Degradation Pathway”, Musial, A. et al the Journal of Biological Chemistry, pp. 24268-24273, 2001, revealed proteolytic inhibitors for iNOS along the proteasome degradation pathway in HEK293 cells, a human epithelial kidney cell line, which had been transfection with the iNOS gene. Investigation revealed the accumulation of iNOS protein in the transfected HEK293 cells when inhibitors of the 26S proteasome were added. Inhibitors of other proteolytic processes did not result in an accumulation of iNOS. Degradation of iNOS by the proteasomal pathway was found to be both time and dose dependant based upon the results obtained with specific inhibitors of the 26S proteasome. A small accumulation of iNOS in cells was discovered using lysosomal inhibitors when tested with the transfected HEK292 cells, but this was concluded to be a minor degradation pathway. When the protease inhibitors were tested on LPS induced RAW 264.7 cells, it was concluded that the proteasomal pathway was the major degradation route for iNOS in LPS induced RAW 264.7 cells.

An article entitled “Ubiquitination of Inducible Nitric-oxide Synthase Is Required for its Degradation”’Kolodziejski, P., PNAS Vol. 99 No. 19 pp. 12315-12320, 17 September 2002, found that ubiquintination of iNOS was required for proteasomal degradation of the protein in three different types of cultured cells. Further, through the use of a mutant cell line that produced a thermolabile ubiquintin-activating enzyme, it was shown that iNOS must be ubiquintinated prior to its degradation by the 26S proteasome.

In an article entitled “TGF-beta1 Enhances Degradation of the IFN-γ-induced iNOS Protein Via Proteasomes in RAW 264.7 Cells”, Nitric Oxide pp. 78-87, August 2005; it was indicated that the treatment of induced RAW 264.7 cells with TGF-β1 resulted in a decrease in the amount of iNOS contained in the cells. It was also shown that the blocking of the proteasomal degradation pathway with an inhibitor resulted in an accumulation of iNOS in the IFNγ-induced RAW 264.7 cells. It was proposed that post-translational regulation of iNOS was responsible for the changes observed which suppressed the amount of NO produced during inflammatory processes.

U.S. patent application Ser. Nos. 10/849,768 and 11/129,452 propose therapeutic agents for iNOS in order to alleviate the sepsis, severe sepsis, or septic shock pathology in a mammalian subject, and U.S. patent application Ser. No. 11/208,143 describes a mammalian model of sepsis that can be used to evaluate potential therapeutic treatments of SIRS, sepsis, severe sepsis, or septic shock in a mammalian subject.

Although the detection of iNOS in the blood of patients may greatly aid in the treatment of those patients by conventional therapies and although other potential therapeutics have been proposed and tested, the efficacy of the potential therapeutics in treating humans has not yet been established.

An improved therapy for the treatment of sepsis, severe sepsis, and septic shock would be a notable advance in the medical field.

### BRIEF SUMMARY OF THE INVENTION

In accordance with the present invention a novel and useful therapeutic agent for the treatment of an illness in a mammalian subject where iNOS is present is herein provided.

Through the realization that the particulate fraction of iNOS, with or without LPS priming, may function as a signal of death in mammalian subjects, experiments were conducted in mice to ascertain the existence of an agent to decrease or stop the conversion of soluble iNOS to particulate iNOS.

Since the conversion to particulate iNOS was due, at least in part, to proteolytic cleavage, inhibiting or stopping the cleavage of iNOS was investigated in relation to the lethality produced by the iNOS particulate fraction. In this regard, several protease inhibitors were found to greatly reduce the proteolytic cleavage of iNOS and, thereby, the lethality of the particulate iNOS fraction in mammalian subjects. The effectiveness of the protease inhibitors was confirmed by in vivo testing of the particulate fractions obtained in the presence of protease inhibitors on mammalian subjects using a mouse model of sepsis previously proposed. As a result, the protease inhibitors leupeptin and E-64 were found to stop the cleavage of iNOS in situ and to prevent death in mammalian subjects challenged with the particulate fraction of iNOS in which the proteolytic cleavage of iNOS in situ had been dramatically decreased.

It may be apparent that a novel and useful therapeutic agent for treatment of an illness is herein provided.

It is therefore a primary object of the present invention to provide a therapeutic agent and method for treatment of a mammalian illness where iNOS is present.

Another object of the present invention is to provide a therapeutic agent for the treatment of systemic inflammatory response syndrome (SIRS), sepsis, severe sepsis, or septic shock in mammalian subjects.

A further object of the present invention is to provide a therapeutic agent which prevents death in mammalian subjects through the action(s) of the soluble fraction of iNOS.

The invention possesses other objects and advantages which will become apparent as the specification continues.

### BRIEF DESCRIPTION OF THE SEVERAL VIEWS OF THE DRAWING

FIG. 1 is a photograph showing a field of peripheral blood mononuclear cells (PBMCS) from a septic patient, in which iNOS-containing vesicles (presumably apoptotic bodies) are separate from the cells, at 200×.

FIG. 2 is an image of a western immunoblot showing the supernatant (soluble) fraction and particulate fraction of iNOS where, lanes 1 and 7 are molecular weight standards, lane 2 is the induced supernatant (soluble) fraction at 5 μl, lane 3 is the induced soluble fraction at 2.5 μl, lane 4 is the induced particulate fraction at 5 μl, lane 5 is the induced particulate fraction at 2.5 μl, and lane 6 is an iNOS standard.

FIG. 3 is a chart illustrating the 48 hour survival of mice primed with LPS and four hours later administered the chemical entities described in Example I.

FIG. 4 is a chart illustrating the seven day survival of mice primed with LPS and four hours later administered certain chemical entities, described in Example II.

FIG. 5 is a chart illustrating the seven day survival of mice primed with LPS and four hours later administered certain chemical entities, described in Example III.

FIG. 6 is an image of a Western blot that shows molecular weight standards (lane 1); intact iNOS contained in the high speed supernatant (lanes 2 and 3); intact iNOS and two fragments of iNOS contained in the low speed particulate fraction (lanes 4 and 5); and no iNOS contained in the high speed particulate fraction as described in Example IV.

FIG. 7 is a chart summarizing the results of Example V demonstrating the lethality of particulate iNOS with or without LPS priming of the challenged mice and the ability of anti-iNOS monoclonal antibody to rescue mice that would otherwise die.

FIG. 8 is an image of a Western blot showing the results obtained with the various protease inhibitors described in Example VII, where lane 1 is the particulate fraction with no protease inhibitor added; lanes 2-7 are with aprotinin, α1-protease inhibitor, soybean trypsin inhibitor, E-64, leupeptin, and pepstatin-A, respectively, added to the culture medium three hours after the start of the induction process. The absence of the lower molecular weight bands in lanes 5 and 6 indicate that only E-64 and leupeptin inhibited the proteolytic cleavage of the intact iNOS protein.

FIG. 9 is an image of a Western blot showing the results obtained with the two different protease inhibitors (soybean trypsin and E-64) when they were used in scaled up production runs in order to produce enough of each particulate fraction of iNOS to be tested in our mouse model of sepsis as described in Example IX, where lane 1 is the particulate fraction with no protease inhibitor added; lane 2 is with soybean trypsin inhibitor added; and lane 3 is with E-64 protease inhibitor added. The loss of lower molecular weight fragments (bands in the Western blot) found in the E-64 added particulate fraction (lane 3), as compared to the no inhibitor added and the leupeptin added, respectively, particulate fractions (lanes 1 and 2) provides evidence that E-64 is inhibiting the proteolytic cleavage of intact iNOS protein.

FIG. 10 is a chart summarizing the results of Example IX demonstrating the ability of protease inhibitor E-64 to decrease the lethality of the particulate fraction by inhibiting the fragmentation of iNOS and statistically significant differences were calculated by Student's T-test for mice challenged with E-64 particulate fraction as compared to particulate fraction with no inhibitor added (P<0.0002) and to soybean trypsin inhibitor particulate fraction (P<0.002).

For a better understanding of the invention reference is made to the following detailed description of the preferred embodiments thereof which should be taken in conjunction with the prior described drawings.

### DETAILED DESCRIPTION OF THE PREFERRED EMBODIMENTS OF THE INVENTION

Various aspects of the present invention will evolve from the following detailed description of the preferred embodiments thereof and examples which should be taken together with the hereinbefore described drawings.

In clinical trials, more than 340 human subjects were enrolled, and over 1,200 blood samples were collected and analyzed to determine if the chemilumenescent EIA for iNOS described in U.S. Pat. No. 6,531,578 and U.S. patent application Ser. No. 09/628,585 prognosticated the onset of sepsis and monitored the course of the pathology. It was found that free iNOS (soluble iNOS) was present in the blood samples. Also, particulate iNOS, in the form of membrane associated iNOS, vesicle-associated iNOS, or iNOS in association with another protein (by protein-protein interaction), was present in some of the blood samples. The vesicles that contained the particulate iNOS were not attached to any of the cells.

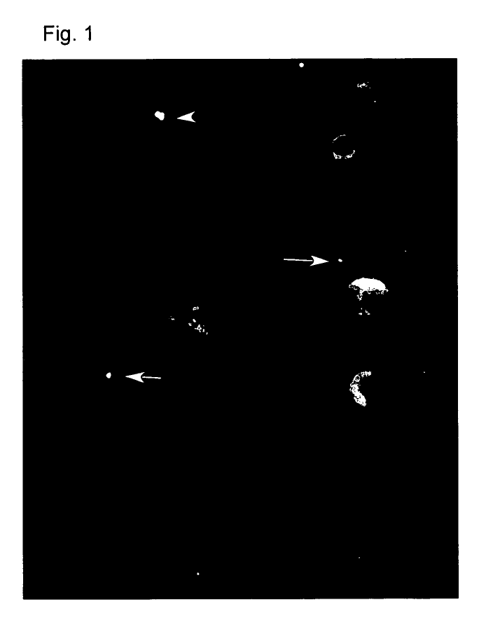

FIG. 1 shows the presence of human iNOS in peripheral blood mononuclear cells (PBMCs) and in vesicles/globules that are not cell associated. In FIG. 1 small extra cellular vesicles, presumably apoptotic bodies, appear as white dots and are separate from the cells (arrows). The immunostaining for iNOS with the anti-iNOS monoclonal antibody 2A1-F8 appears granular in the PBMCs. The anti-iNOS monoclonal antibody 2A1-F8 is disclosed in U.S. Pat. No. 6,531,578. FIG. 1 provides evidence for the existence of apoptotic bodies in vivo in humans. Particulate or vesicle-associated iNOS was only found in samples from patients afflicted with sepsis, severe sepsis, or septic shock. The presence of apoptotic bodies as revealed in FIG. 1 in the blood stream of a human was theorized to be an indication of the presence of sepsis or an indication of the severity of the pathology.

Since soluble iNOS and the particulate or vesicle-associated iNOS are only found in the blood of critically ill patients, the contribution to the pathology of systemic inflammatory response syndrome (SIRS), sepsis, severe sepsis, or septic shock by these forms of iNOS was investigated. In other words, the presence of soluble or particulate iNOS in the circulation was theorized to be deleterious to patients with sepsis or pre-sepsis. Consequently, it was reasoned that inhibition of the production of the iNOS that is found in the blood stream of patients with sepsis or pre-sepsis conditions may constitute a possible therapeutic treatment for such illnesses.

In order to gather data that might confirm such hypothesis, a mouse model of sepsis was used in a series of experiments. Tests were employed to determine (1) whether or not proteolytic cleavage of intact iNOS contributed to the pathology of systemic inflammatory response syndrome sepsis, severe sepsis, or septic shock and (2) whether or not inhibition of the proteolytic cleavage of iNOS with specific protease inhibitors would be a possible therapeutic treatment for these life threatening conditions.

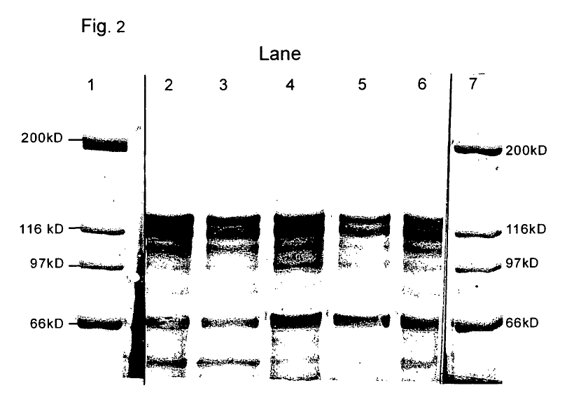

DLD-1-5B2 cells were induced to produce human iNOS by the addition of a mixture of cytokines. In an article entitled “Transcriptional Regulation of Human Inducible Nitric Oxide Synthase Gene In An Intestinal Epithelial Cell Line”, Linn et al, Am J Physiol, 272: G1499-G1508 (1997), it was shown that DLD-1 cells can be induced to produce human iNOS. The induced cells were lysed, and two types of iNOS were isolated by centrifugation, a supernatant (soluble) iNOS fraction and a particulate iNOS fraction. FIG. 2 shows a Western immunoblot which indicates that the pooled supernatant (soluble) fraction of induced DLD-1-5B2 (a clone of DLD-1) cells contained iNOS (lanes 2 and 3) at the predicted molecular weight of 131 kD. Also, the particulate fraction of induced DLD-1-5B2 cells likewise contained iNOS (lanes 4 and 5) as shown by the band at 131 kD. Lane 6 contained an iNOS standard and lanes 1 and 7 contained standard proteins used as molecular weight markers at the indicated weights. In order to produce and isolate the supernatant (soluble) fraction and the particulate fraction of iNOS from induced DLD-1-5B2 cell cultures, the following steps were followed:

1. DLD-1-5B2 cells were grown in culture starting from frozen cryopreserved cells;

2. The expression of iNOS was induced in the cells;

3. The induced cells were harvested; and

4. The iNOS in the induced cells was isolated and fractionated into soluble and particulate fractions.

Briefly, cryopreserved cells were transferred into a T-75 flask containing DLD-1-5B2 medium (90 percent DMEM and 10 percent FBS supplemented with Pen/Strep), and cultured in a humidified atmosphere of 5 percent CO₂ in air at 37° C. The culture medium was changed every other day until the cells were almost confluent. Subsequently, the medium was changed daily until the cell cultures were either split or induced. When the DLD-1-5B2 cells were near confluence and in log-phase growth, the cells were split 1:6 to 1:10 into additional T-75 flasks.

DLD-1-5B2 cells, two days past confluence, were induced to express human iNOS using a mixture of recombinant human gamma interferon (rhIFNγ) at 8.33 ng/ml, recombinant human tumor necrosis factor-alpha (rhTNFα) at 3.3 ng/ml, and recombinant human interleukin-1β (rhIL-1β) at 3.3 ng/ml for 12-18 hours before being washed and harvested with trypsin/EDTA. The induced cells were washed three times with sterile PBS, transferred into sterile tubes in a small volume of sterile PBS, and stored frozen at −20° C. until processed for the isolation and fractionation of iNOS.

The frozen, induced DLD-1-5B2 cells were thawed in an ice water bath and lysed by two rapid freeze/thaw cycles using dry ice. The iNOS produced by the induced DLD-1-5B2 cells was then fractionated into a supernatant (soluble) fraction and a particulate fraction. The lysed cells were centrifuged at 16,000×g at 4° C. for 30 minutes to pellet the particulate fraction. The supernatant fraction that contained the soluble iNOS was transferred and stored at −20° C. or used fresh. The pellets were washed once, resuspended in a small volume of ice cold sterile PBS, and the resulting particulate fraction of iNOS was stored at −20° C. or used fresh.

In general, once the cryopreserved cells were thawed and placed in culture medium, they reach log phase growth after a few days at 37° C. in a humidified 5 percent CO₂/95 percent air atmosphere. DLD-1 cells are available from ATCC (CAT.#CCL221). The DLD-1-5B2 cell line was derived by sub-cloning the DLD-1 cells using standard cloning techniques.

A mammalian model of sepsis, severe sepsis, and septic shock has been developed and tested. In particular, tests were employed to determine whether or not the supernatant and/or the particulate iNOS fractions contributed to the pathology of sepsis, severe sepsis, or septic shock. In order to gather data that might confirm such hypotheses, a mammalian model of sepsis, utilizing mice, was used in a series of experiments. The effects of the supernatant (soluble) iNOS fraction and of the particulate iNOS fraction were tested on mice primed (1) with a sub-lethal dose of the innate immune system activator LPS or (2) without priming (a saline injection) as a model of sepsis. This was done to determine the effects that the two different fractions of iNOS protein had on the mice. The results of these experiments led to the discovery that the iNOS protein functions in signaling death in the mice. Several experiments were conducted, and it was discovered that iNOS in the particulate fraction, rather than the supernatant fraction of iNOS, plays a role in causing death in this mouse model of sepsis. It was also found that the particulate or vesicle associated iNOS alone was lethal and did not require a priming dose of an innate immune system activator for it to be lethal. However, priming with an innate immune system activator augmented the effect, and thereby, required a lower dose of the particulate iNOS for lethality. Administration of the particulate iNOS fraction to non-primed mice could result in almost immediate death depending upon the dose administered. It was concluded that particulate iNOS was believed to be responsible for the lethal effect observed both in LPS primed mice and non-primed mice.

In summary, the effects of the supernatant iNOS fraction and of the particulate iNOS fraction were tested on non-primed mice and on mice primed with a sub-lethal dose of LPS as a model of sepsis. This was done to determine the effects the two different fractions of iNOS protein had on the viability of the mice. The results of these experiments led to the discovery that the iNOS protein has a function in signaling death in the mice. Several experiments were conducted, and it was discovered that iNOS in the particulate fraction, rather than supernatant (soluble) fraction of iNOS, plays a role in causing death in this mouse model of sepsis. It is also found from these experiments that LPS priming of mice was not necessary for the effect of the particulate iNOS to be exerted. The administration of the particulate iNOS fraction to non-primed mice caused almost immediate death. Consequently, particulate iNOS is believed to be responsible for the lethal effect observed in LPS primed mice.

It was reasoned that the conversion of soluble iNOS to particulate iNOS, and thereby to lethal iNOS, might be the result of the proteolytic cleavage of the intact protein molecule. It was hypothesized that decreasing the cleavage process might be beneficial to septic patients since the lethal form of iNOS would be reduced or eliminated. This hypothesis was tested using various protease inhibitors. The loss of lethality produced by specific protease inhibitors was assessed by testing the lethal effects exerted by the administration of the particulate iNOS produced in the presence and absence of protease inhibitors to LPS primed mice. It was found that different protease inhibitors varied in their individual ability to reduce the lethality of particulate iNOS in the mouse model of sepsis. Protease inhibitors that did not decrease the cleavage of the iNOS protein, such as α-1-protease inhibitor (α-1-PI), soybean trypsin inhibitor (SBTI), and pepstatin-A, had no effect on the lethality of the particulate iNOS fraction. However, Calpain I inhibitors such as the protease inhibitor E-64, and leupeptin were found to decrease the proteolytic cleavage of iNOS, and decrease the lethality of the particulate iNOS fraction thereby. Based upon these results analogs of protease inhibitors E-64 and leupeptin are also believed to inhibit or decrease the cleavage of iNOS.

The following examples are provided to further illustrate the present invention but are not deemed to limit the invention in any manner.

### EXAMPLE I

The two fractions of human iNOS, illustrated in FIG. 2, were investigated as to their effect on LPS primed mice as an animal model of sepsis. Prior to starting the experiment, soluble iNOS was removed from the supernatant fraction by selective absorption onto MAG-BEADS coated with one or more of the anti-iNOS MAbs found in the U.S. Pat. No. 6,531,578. Briefly, MAG-BEADS covalently bonded to goat anti-mouse IgG IgG were purchased from the Pierce Chemical Co. in Rockford, Ill. Culture supernatant containing secreted anti-iNOS MAbs from clones 21C10-1D10, 2A1-F8, 1E8-B8 and 2D2-B2 were applied individually to aliquots of the suspended MAG-BEADS in order to load the MAG-BEADS with monoclonal antibodies specific for iNOS. The supernatant fraction containing soluble iNOS was diluted 1:2 and applied to pooled, washed, and resuspended anti-iNOS coated MAG-BEADS. The suspension was incubated overnight with gentle mixing to allow the soluble iNOS to bind to the anti-iNOS MAbs coated onto the MAG-BEADS before the tube containing the suspension was placed onto a magnetic rack. All the beads congregated on the sides of the tube next to the magnets. The resulting iNOS-depleted supernatant fraction was transferred and diluted to a final volume to yield a 1:5 dilution as compared to the stock supernatant fraction. A 1:5 dilution of the stock iNOS supernatant fraction was also prepared in sterile saline. Samples of the 1:5 iNOS-depleted supernatant fraction, of the 1:5 diluted stock supernatant fraction, and of the iNOS coated MAG-BEADS used to create the iNOS-depleted supernatant fraction were all analyzed to determine if the soluble iNOS had been removed and to demonstrate that the soluble iNOS was bound to the anti-iNOS MAbs attached to the MAG-BEADS. These analyses showed that more than 90 percent of the soluble iNOS had been removed from the supernatant (soluble) fraction (iNOS-depleted supernatant fraction), and that the iNOS was bound to the MAG-BEADS which had been loaded with the anti-iNOS MAbs.

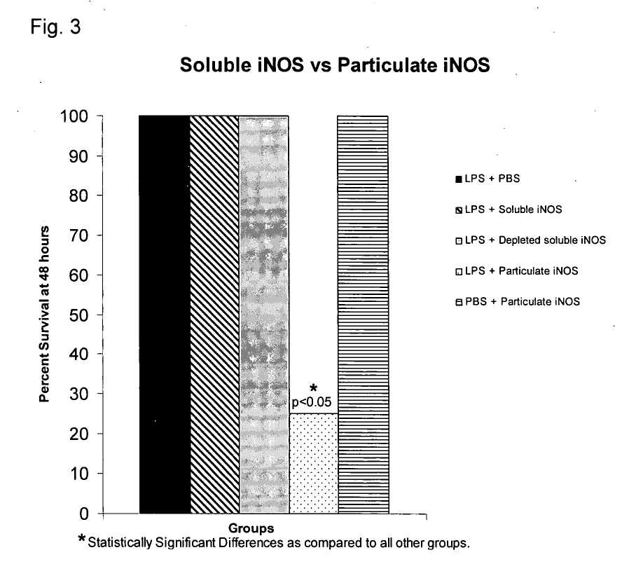

Groups of mice containing both genders were injected IP with sterile saline only, or with a sub-lethal dose of LPS (2 mg/kg body weight of LPS Serotype 0111:B4 from E. coli, obtained from Sigma Chemical Co., Saint Louis, Mo.) in sterile saline. After four hours, only the mice injected with LPS became lethargic and developed diarrhea. The saline or LPS-primed mice were then given a tail vein injection of either saline or one of the following: the supernatant fraction containing soluble iNOS (soluble iNOS), the supernatant fraction depleted of iNOS (iNOS-depleted soluble fraction), or a suspension of particulate iNOS produced by and isolated from induced DLD-1-5B2 cells. FIG. 3 shows the results of this experiment. None of the saline-primed mice showed any effect with any of the test samples. No effect was seen with the LPS-primed mice upon administration by tail vein injection of either a dose of saline, a dose of the supernatant fraction of iNOS, or a dose of iNOS-depleted supernatant fraction. However, when the particulate fraction of iNOS was administered to the LPS prime mice, all the mice died almost immediately. In this series of experiments, no effect was seen on the saline-primed mice given the same dose of particulate iNOS. It was concluded that the particulate fraction of iNOS, but not the supernatant fraction of iNOS which contained soluble iNOS, when administered to LPS-primed mice caused an almost immediate death.

### EXAMPLE II

The anti-human iNOS MAbs found in U.S. Pat. No. 6,531,578 were employed in order to investigate the inhibition of the killing effect seen with the particulate fraction of human iNOS in LPS-primed mice. Particulate iNOS was removed from the particulate fraction by selective absorption onto MAG-BEADS coated with the anti-iNOS MAbs as described in Example I. A similar procedure to the one described with respect to depletion of the supernatant fraction in Example I was employed. Samples of the iNOS-depleted particulate fraction, of the stock particulate fraction, and of the iNOS loaded MAG-BEADS used to create the iNOS-depleted particulate fraction, were analyzed to show that the particulate iNOS had been removed. Our analyses showed that more than 90 percent of the particulate iNOS had been removed from the particulate fraction (iNOS-depleted particulate fraction) and that the iNOS was bound to the MAG-BEADS which had been loaded with the anti-iNOS MAbs.

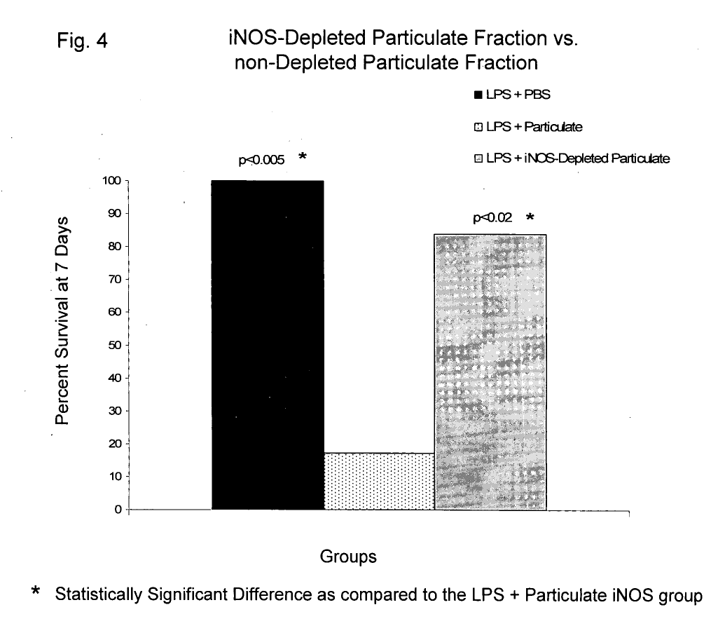

The effect that the iNOS-depleted particulate fraction had on the LPS-primed mice was compared to that seen with the stock (non-depleted) particulate fraction containing particulate iNOS. Groups of mice containing both genders were injected IP with a sub-lethal dose of LPS (2 mg/kg body weight of LPS serotype 0111:B4 from E. coli obtained from the Sigma Chemical Company) in sterile saline. After four hours, all the mice primed with LPS became lethargic and developed diarrhea. The various groups of mice were then given a tail vein injection of either saline, stock particulate iNOS at a 1:10 dilution, or iNOS-depleted particulate fraction at a 1:10 dilution as compared to the starting stock suspension. FIG. 4 represents these definitive results. None of the mice that received a priming IP injection of LPS followed four hours later by a tail injection of saline showed any effect since they all survived seven days until the end of the experiment of this Example. However, only 17 percent (one out of six) of the mice that received a priming IP injection of LPS followed four hours later by a tail injection of particulate iNOS at a 1:10 dilution, survived for seven days. Significantly, 84 percent (five of six) of the LPS-primed mice that received a tail vein injection of the iNOS-deleted particulate fraction survived for seven days. When these data were compared, a high degree of statistically significant difference was found between the survival of the mice administered the particulate iNOS fraction and those administered saline (P<0.005 by Student's T-test) or the iNOS-depleted particulate fraction (P<0.02). There was no statistically significant difference between the LPS-primed mice that received a saline IV injection and those that received the iNOS-depleted particulate fraction. Thus, the specific removal of the particulate iNOS from the particulate fraction abolished the lethal effect seen in the LPS-primed mice that received the particulate iNOS fraction. It was concluded that (1) particulate iNOS by itself or particulate iNOS in association with one or more proteins was responsible for the lethal effect observed in LPS-primed mice and (2) removal of the particulate iNOS or particulate iNOS in association with one or more proteins, by absorption from solution using immobilized anti-iNOS MAbs, stopped the lethal effects asserted by the administration of the particulate iNOS.

### EXAMPLE III

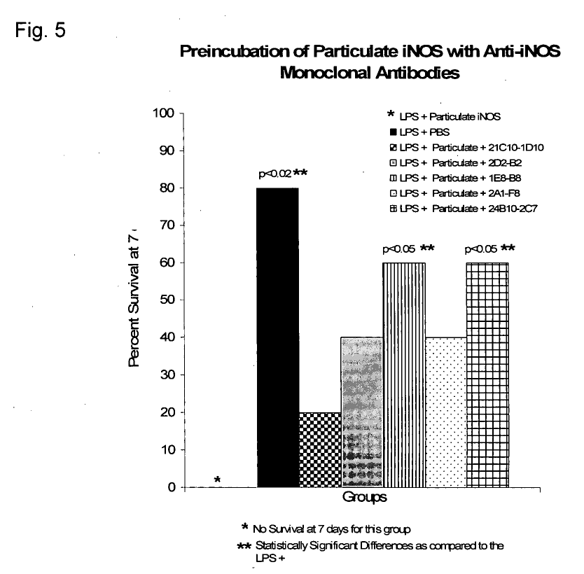

A second method was employed to study the ability of the anti-human iNOS MAbs of U.S. Pat. No. 6,531,578 to inhibit the killing effect seen with the particulate fraction of human iNOS in LPS-primed mice as a model for sepsis. Instead of physically removing the particulate iNOS from the particulate fraction as was performed in Example II, individual anti-iNOS MAbs contained in ascites fluid were added directly to aliquots of the particulate fraction that contained particulate iNOS. The proteins contained in the particulate iNOS fraction were allowed to bind to the anti-iNOS MAbs for 45 minutes before the material was injected IV into mice. Five different anti-iNOS MAbs were tested for their individual ability to inhibit (neutralize) the killing effect of the particulate human iNOS. Groups of mice were primed with a sub-lethal dose of LPS (2 mg/kg body weight of LPS Serotype 0111:B4 from E. coli obtained from the Sigma Chemical Company) in sterile saline. After four hours all the LPS-primed mice became lethargic and developed diarrhea. The various groups of mice were given a tail vein injection of either saline, stock particulate iNOS at a 1:10 dilution, or stock particulate iNOS at a 1:10 dilution that had been preincubated for 45 minutes with one of five different anti-iNOS MAbs. Each of the five different anti-iNOS MAbs was used at a 1:50 dilution of the ascites fluid. The results varied and are shown in FIG. 5. All the LPS-primed mice that received a tail vein injection of the stock particulate iNOS diluted 1:10 in sterile saline died within the first 24 hours of the seven day experiment. In contrast, four out of five (80 percent) of the LPS-primed mice administered a saline tail vein injection survived seven days (P<0.02). The ability of the anti-iNOS MAbs to neutralize the lethal effect of the particulate iNOS varied depending on the MAb being tested. Of the five different anti-iNOS MAbs tested, anti-iNOS MAb 1E8-B8 and 24B10-2C10 were the best at neutralizing the lethal effects of the particulate iNOS on LPS-primed mice. In both cases, three out of five mice survived seven days (P<0.05). Two other anti-iNOS MAbs (2D2-B2 and 2A1-F8) were also somewhat effective in stopping the mice from dying, i.e. two out of five mice in each of these groups survived seven days. One anti-iNOS MAb (21C10-1D10) was much less effective since only one out of five mice survived seven days. It was concluded (1) that it is not necessary to remove physically the particulate iNOS from the solution in order to neutralize its lethality; (2) that anti-iNOS MAbs can neutralize the lethal effects of particulate iNOS on LPS-primed mice by binding to iNOS or by binding to the protein-protein complex containing particulate iNOS; and (3) that different anti-iNOS MAbs vary in their individual ability to neutralize the lethality of particulate iNOS either as particulate iNOS itself or in association with one or more proteins.

### EXAMPLE IV

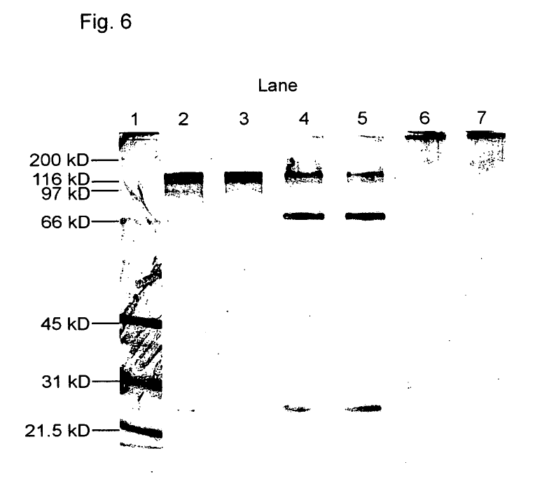

Experiments were performed to determine if the iNOS contained in the particulate fraction is located in the cellular membrane, or in vesicles, or in other structures, such as membrane fragments. Cytokine induced DLD-1-5B2 cells were lysed by hypotonic shock instead of by 2 freeze/thaw cycles in order to rupture the cellular membrane and to release their cellular components and membrane structures, including blebbing vesicles, into the solution and, thereby, avoid denaturing the proteins, as can occur with multiple freeze/thaw cycles. The solution containing the lysed cells was subjected first to low speed centrifugation (at 300×g) to obtain a low speed particulate pellet. The low speed supernatant was then subjected to higher speed centrifugation (at 16,000×g) to produce both a high speed particulate pellet and a high speed supernatant. When these three fractions were tested for their killing activity in LPS-primed mice, the lethal activity was only found in the low speed particulate fraction. Neither the high speed pellet nor the high speed supernatant was lethal when injected intravenously into LPS-primed mice. When the low speed particulate fraction was examined microscopically, two types of structures were observed. One was the cellular membrane of “ghost” cells, i.e. ruptured cell remnants, and the other was small vesicles which many times appeared linked together like beads on a string. When this preparation was stained by immunofluorescence using the anti-iNOS MAbs of U.S. Pat. No. 6,531,578 to immunolocalize the iNOS, intense fluorescent staining was observed exclusively in the small vesicles. No IFA staining of iNOS was observed in the “ghost” cells or in any other structure. The size of these vesicles (apoptoic bodies) and their intense IFA staining with the noted anti-iNOS MAbs is very similar, if not identical, to that observed in the blood of human septic patients (FIG. 1). When these preparations were also analyzed by Western blot after SDS-PAGE separation of the proteins (FIG. 6), the high speed supernatant was found to contain intact iNOS. The low speed particulate fraction contained intact iNOS, but it also contained two iNOS fragments that bound the anti-iNOS MAb 2D2-B2 used in these experiments (FIG. 6). It was repeatedly found that the high speed supernatant does not kill the challenged mice in the animal model of sepsis of Examples I-III while the particulate fraction whether produced by freeze/thaw cycles or by hypotonic shock is lethal to the challenged mice.

### EXAMPLE V

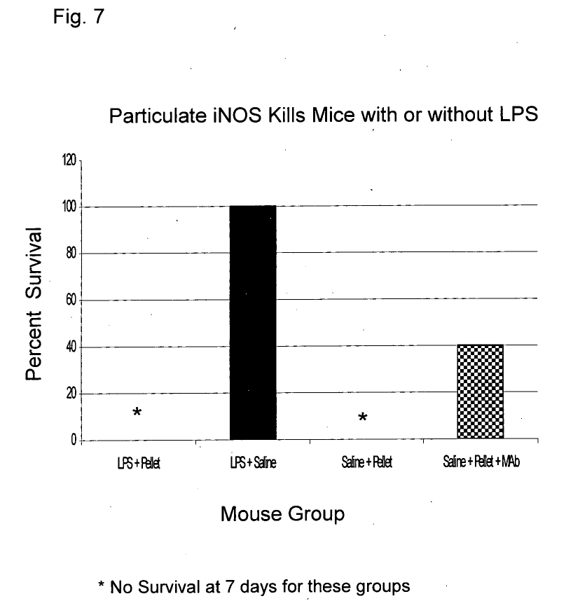

In a series of experiments particulate iNOS, with or without LPS priming, was determined to kill mice, and treatment with anti-iNOS MAb was deemed capable of rescuing some mice that would otherwise have died. In these experiments mice were primed with LPS or saline for four hours before being challenged with either saline, particulate or vesicle associated iNOS isolated from induced DLD-1-5B2 cells, or particulate or vesicle associated iNOS that had been preincubated for 30 minutes with anti-iNOS MAb 2A12-A4. The particulate or vesicle associated iNOS killed all five mice in both the LPS primed group and the group that had only been primed with saline. When the particulate iNOS challenge was replaced by a saline challenge, after being primed with LPS, all five mice in the group lived. These data showed (1) that the dose of LPS used to prime the mice was not lethal and (2) that challenging the mice with particulate or vesicle associated iNOS with or without LPS priming resulted in death by sepsis. If the particulate or vesicle associated iNOS was pretreated with anti-iNOS MAb, 40 percent of the mice (two of five) were rescued from death by sepsis. This showed that the anti-iNOS MAb 2A12-A4 is a neutralizing antibody and might be useful for therapeutic treatment of sepsis. The effectiveness of other potential treatments for sepsis can be evaluated using this animal model. FIG. 7 depicts the result of this example, and particulate iNOS is labeled “pellet” in FIG. 7.

### EXAMPLE VI

The effectiveness of particulate iNOS in killing mice after priming with either LPS or saline was investigated in a number of experiments using different doses of particulate iNOS. Mice were primed with either LPS or sterile saline four hours prior to the challenge with particulate iNOS. Cumulative data indicated that of the 35 total mice in both groups, 24 died in the LPS primed group i.e. 68.6 percent died and 31.4 percent survived, and that 20 died in the saline primed group, i.e. 57.1 percent died and 42.9 percent survived. Statistical analysis of these data show that no significant difference exists between these two groups which means that particulate or vesicle associated iNOS alone is just as lethal as particulate or vesicle associated iNOS administered after a priming dose of the innate immune system activator, lipopolysaccharide (LPS).

### EXAMPLE VII

In two series of experiments, (1) humanized MAb 1E8-B8 immobilized on Mag-beads, (2) peptide G-11 to which MAb 1E8-B8 binds, and (3) the particulate fraction obtained from cytokine induced DLD-1-5B2 cells that were lysed either by 2 freeze/thaw cycles or by hypotonic shock were used to recover the hiNOS and associated materials including vesicles bound to the anti-hiNOS loaded MAG-BEADS. After treating the two particulate fractions with humanized MAb 1E8-B8 immobilized on MAG-BEADS, the MAG-BEADS were washed to remove unbound material. The material bound to the anti-hiNOS loaded MAG-BEADS were competed-off the humanized anti-hiNOS MAb by incubating the loaded beads with a high concentration (100 μg) of peptide G-11. The material competed-off the humanized anti-hiNOS 1E8-B8 MAb bound to the MAG-BEADS was centrifuged, washed and used to challenge mice primed with a sub-lethal dose of LPS in our mouse model of sepsis. Both samples were found to possess the lethal activity initially discovered in the particulate fraction obtained from cytokine induced and lysed DLD-1-5B2 cells. Namely, with the recovered sample from the particulate fraction prepared by 2 freeze/thaw cycles, 2 out of 2 mice died, and with the recovered sample from the particulate fraction prepared by hypotonic shock, 1 out of 2 mice died. When the proteins contained in the material competed-off the anti-hiNOS loaded MAG-BEADS were analyzed by Western blots using anti-hiNOS MAb clone 2D2-B2 that binds to hiNOS[781-798], three main bands were found at apparent molecular weights of 131kD, 70 kD, and 27 kD for the material recovered from the particulate fraction prepared by 2 freeze/thaw cycles. A single band at 70 kD was found for the sample recovered from the low speed particulate fraction prepared by hypotonic shock.

### EXAMPLE VIII

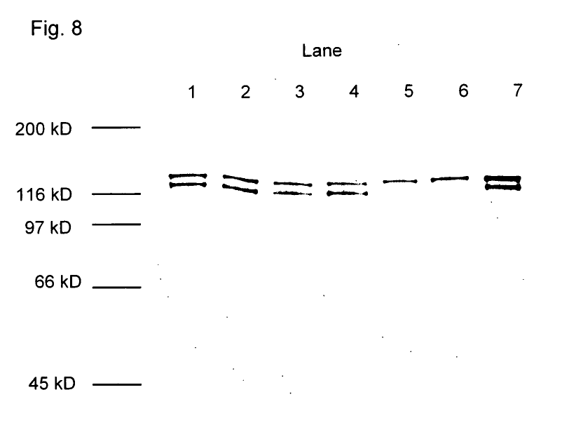

The ability of six specific protease inhibitors to inhibit the cleavage of iNOS in situ was investigated using induced DLD-1-5B2 cells cultured in 24-well plates. Briefly, DLD-1-5B2 cells were cultured in 24-well plates instead of T-75 flasks, as described previously. Two days past confluence, the cells were induced with the same mixture of cytokines, but after three hours, the induction medium was removed from each well, the cells washed with pre-warmed sterile saline, and the medium replaced either with fresh DMEM, or with DMEM containing one of the six protease inhibitors being tested. Each inhibitor was assayed in triplicate at 20 μg/well. Nine hours later (12 hours after the start of the induction) the medium containing the inhibitors was removed, the cells were washed three times with ice-cold PBS, and the 24-well plates were placed on sheets of ice for 10 minutes to loosen the cells from the bottom of the plastic wells. The cells were blown off each well individually using a mechanical pipettor, sterile pipet tips, and ice-cold, sterile PBS. The cells were transferred to individual microfuge tubes on ice, collected by centrifugation, and washed twice with sterile, ice-cold PBS. No trypsin or other protease or EDTA was used during the harvesting or processing of the induced cells. The cells were lysed by 2 freeze/thaw cycles using dry ice and processed as previously described to isolate a supernatant fraction and a particulate fraction by centrifugation at 16,000×g. Both fractions were assayed in duplicate using the chemiluminescent EIA taught in U.S. patent application Ser. No. 09/628,585, and pools of the triplicate samples obtained for each protease inhibitor were analyzed by western blot. No statistically significant difference was found in the amount of iNOS contained in any of the supernatant fractions when compared to other supernatant fractions. Likewise, no statistically significant difference was found in the amount of iNOS contained in any of the particulate fractions when compared to other particulate fractions as measured by the EIA. Further, the distribution of iNOS between the supernatant and particulate fractions was similar for all the samples: the percent of the total iNOS in the supernatant fraction ranged from 55% to 65% and the percent of the total iNOS in the particulate fraction ranged from 35% to 45%. However, as is shown in FIG. 8, a difference in the form of the iNOS was found in the particulate fractions obtained from two of the protease inhibitors. Leupeptin and E-64, stopped (inhibited) the fragmentation of iNOS in situ while the other four protease inhibitors tested, aprotinin, α1-protease inhibitor (α1PI), soybean trypsin inhibitor (SBTI), and pepstatin-A, did not stop (inhibit) this cleavage process.

### EXAMPLE IX

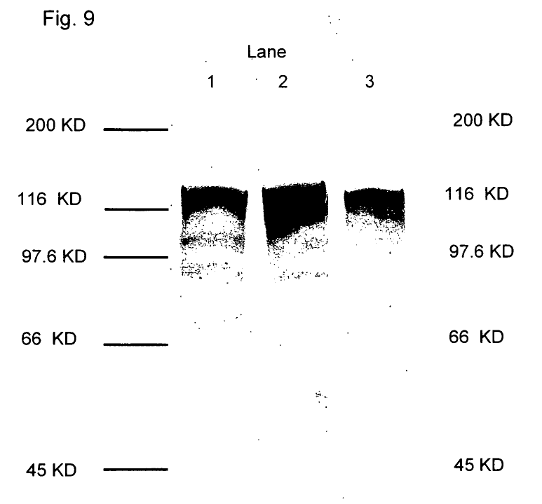

Two of the protease inhibitors tested in Example VIII, E-64 and SBTI which did stop and did not stop, respectively, the cleavage process, were selected for further investigation. The quantity of particulate fraction produced was scaled up in T-75 culture flasks for both of these protease inhibitors in order to make enough material to be tested for their individual lethality in our mouse model of sepsis. As was described in Example VIII, the induction medium was applied to the cell monolayers in T-75 flasks for 3 hours before being removed, the cells washed with pre-warmed sterile saline, and fresh medium applied that contained no inhibitor or that contained one of the two protease inhibitors. Nine hours later the induced cells from each of the three preparation (no inhibitor added, E-64 protease inhibitor added, or SBTI added) were harvested again without the use of trypsin, or any other protease, or EDTA. The induced cells were lysed by 2 freeze/thaw cycles and the supernatant and particulate fractions isolated by centrifugation at 16,000×g. When the three supernatant fractions and the three particulate fractions were assayed by chemiluminescent EIA, no statistically significant difference was found when a supernatant fraction was compared to any other supernatant fraction or when a particulate fraction was compared to any other particulate fraction obtained from these three preparations. However, as with the 24-well experiment described in Example VIII, a difference in the form of the iNOS in the particulate fraction obtained with the E-64 protease inhibitor was found. FIG. 9 is a western blot demonstrating the ability of E-64 protease inhibitor (lane 3) to inhibit the fragmentation of iNOS in situ in the larger scale production and the inability of soybean trypsin inhibitor (lane 2) to inhibit this cleavage process in the larger scale production. In order to visualize minor components and fragments in the three samples, each lane in this Western blot was intentionally overloaded with sample. This resulted in the doublet bands found in lanes 1 and 4 of FIG. 8 for the no inhibitor added and the SBTI added, respectively, particulate fractions being fused (i.e. unresolved) on this overloaded western membrane. These results confirmed and extended the observations made with these protease inhibitors when tested on cells grown in 24-well plates.

### EXAMPLE X

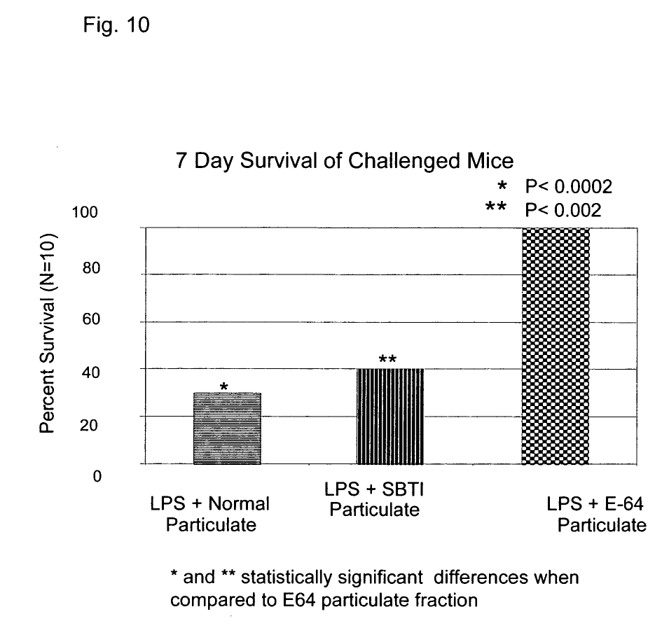

The three particulate fractions prepared in Example IX were tested for their lethality on groups of mice (5 female and 5 male mice per group, N=10) using our mouse model of sepsis. When the particulate fraction was isolated from cells induced in the presence of E-64, a statistically significant decrease in lethality was observed as compared to the particulate fractions produced in the absence of any protease inhibitor (Normal Particulate, P<0.0002 by Student's T-test) or in the presence of SBTI (P<0.002). Thus, the ability of the E-64 protease inhibitor to stop the cleavage of the iNOS protein resulted in the particulate fraction being dramatically less lethal when assayed in this animal model of sepsis (FIG. 10). It is believed that treatment of septic patients with a protease inhibitor that decreases or stops the cleavage of iNOS in vivo could serve as an effective therapy for SIRS, sepsis, severe sepsis and septic shock.

While in the foregoing, embodiments and Examples representing the carrying out of the present invention have been set forth in considerable detail for the purposes of making a complete disclosure of the invention, it may be apparent to those of skill in the art that numerous changes may be made in such detail without departing from the spirit and principles of the invention.

What is claimed is:

1. A therapeutic agent for the treatment of an illness in a mammalian subject in which inducible nitric oxide synthase (iNOS) is present, comprising:
a protease inhibitor decreasing the proteolytic cleavage of the inducible nitric oxide synthase (iNOS).

2. The therapeutic agent of claim 1 in which said protease inhibitor comprises a calpain I inhibitor.

3. The therapeutic agent of claim 2 in which said protease inhibitor comprises leupeptin or analog thereof.

4. The therapeutic agent of claim 2 in which said protease inhibitor comprises E-64 or analog thereof.

5. The therapeutic agent of claim 1 which further comprises a protease inhibitor that decreases the proteolytic cleavage of the inducible nitric oxide synthase (iNOS) in vivo.

6. A therapeutic agent for the treatment of systemic inflammatory response syndrome (SIRS), sepsis, severe sepsis, or septic shock in a mammalian subject in which inducible nitric oxide synthase (iNOS) is present, comprising:
a protease inhibitor decreasing the proteolytic cleavage of the inducible nitric oxide synthase (iNOS).

7. The therapeutic agent of claim 1 in which said protease inhibitor comprises a calpain I inhibitor.

8. The therapeutic agent of claim 7 in which said protease inhibitor comprises leupeptin or analog thereof.

9. The therapeutic agent of claim 7 in which said protease inhibitor comprise E-64 or analog thereof.

10. The therapeutic agent of claim 6 in which said protease inhibitor reducing the proteolytic cleavage of the inducible nitric oxide synthase (iNOS) comprises a protease inhibitor decreasing the proteolytic cleavage of the inducible nitric oxide synthase (iNOS) in vivo.

11. A method of treating an illness in a mammalian subject in which inducible nitric oxide synthase (iNOS) is present, comprising the step of:
introducing a protease inhibitor into the body of the mammalian subject to reduce the proteolytic cleavage of inducible nitric oxide synthase (iNOS).

12. The method of claim 11 in which said protease inhibitor comprises a calpain I inhibitor.

13. The method of claim 12 in which said protease inhibitor comprises leupeptin or analog thereof.

14. The method of claim 12 in which said protease inhibitor comprises E-64 or analog thereof.

15. The method of claim 10 in which the illness is selected from the group consisting of systemic inflammatory response syndrome (SIRS), sepsis, severe sepsis, or septic shock.

16. The method of claim 11 in which said protease inhibitor reduces the proteolytic cleavage of the inducible nitric oxide synthase (iNOS) in vivo.
